(19) United States
(12) Patent Application Publication
(10) Pub. No.: US 2022/0008204 A1
(43) Pub. Date: Jan. 13, 2022

(54) HUMAN IMPLANT

(71) Applicant: Ingrowth Biotech Co., Ltd, Kaohsiung City (TW)

(72) Inventors: Royal Su, Kaohsiung City (TW); Jason Sun, Kaohsiung City (TW); Po-Han Chu, Kaohsiung City (TW)

(21) Appl. No.: 17/084,081

(22) Filed: Oct. 29, 2020

(30) Foreign Application Priority Data
Jul. 8, 2020 (TW) 109123071

(51) Int. Cl. A61F 2/30; A61F 2/44; A61F 2/28; A61B 17/80

(52) U.S. Cl. CPC A61F 2/30767; A61F 2/447; A61F 2/2875; A61C 8/0036; A61F 2002/30985; A61F 2002/30784; A61F 2310/00011; A61B 17/80

(57) ABSTRACT

A human implant includes at least one osteoconductive scaffold and at least one main carrier. The at least one osteoconductive scaffold is made of a metal material, is manufactured by 3D printing, and has at least one connecting portion, at least one separation element, and a proliferation portion. The at least one connecting portion is porous. The at least one separation element is disposed on one of two sides of the at least one connecting portion. The proliferation portion is disposed on one of two sides of the at least one separation element away from the at least one connecting portion, wherein osteoblasts proliferate in the proliferation portion. The at least one main carrier is made of a medical macromolecular material and is mounted to the at least one connecting portion being porous, such that the at least one main carrier is mounted to the at least one osteoconductive scaffold.

### BACKGROUND OF THE INVENTION

### 1. Field of the Invention

The present invention relates to a human implant, and more particularly to a human implant with raised extent of bone fusion and extended life expectancy, having an elastic modulus close to that of the bones in a human body, having a connecting portion with an amount of deformation similar to that of a main carrier of the human implant in all directions, having components mounted securely, and without the problem that the fatigue stress exceeds the endurance limit.

### 2. Description of Related Art

A conventional human implant, such as an interbody fusion cage, a bone plate, an artificial mandible, and an artificial tooth root, mainly includes at least one main carrier, at least one osteoconductive scaffold, and at least one fixation pin. The at least one main carrier is made of a medical macromolecular material having an elastic modulus close to that of the bones of a human body, such that the stress that the at least one main carrier can bear is close to the stress that the bones of the human body can bear. The at least one osteoconductive scaffold is made of a biocompatible metal, and the at least one osteoconductive scaffold induces osteoblasts to grow therein and become fused therewith. The at least one main carrier and the at least one osteoconductive scaffold are mounted together via the at least one fixation pin to form the conventional human implant.

However, the conventional human implant has the following drawbacks.

1. The at least one osteoconductive scaffold made of metal has a higher elastic modulus than that of the at least one main carrier made of medical macromolecular material, so the fatigue stress of the conventional human implant may exceed the endurance limit of the conventional human implant.

2. To mount the at least one main carrier and the at least one osteoconductive scaffold together, it is necessary to insert a fixation element such as the at least one fixation pin through the at least one main carrier and the at least one osteoconductive scaffold to avoid separation of the at least one main carrier and the at least one osteoconductive scaffold when stress is applied on the conventional human implant implanted in the human body.

3. According to the above-mentioned point 2, regardless that the conventional human implant is served as the interbody fusion, the bone plate, or the artificial mandible, the at least one fixation pin tends to detach from the at least one main carrier and the at least one osteoconductive scaffold after a long term use. In such a condition, the at least one fixation pin will remain in the human body. In addition to that, the at least one main carrier and the at least one osteoconductive scaffold will separate and the conventional human implant will lose its original function.

To overcome the shortcomings of the conventional human implant, the present invention tends to provide a human implant to mitigate or obviate the aforementioned problems.

### SUMMARY OF THE INVENTION

The main objective of the present invention is to provide a human implant.

The human implant in accordance with the present invention includes at least one osteoconductive scaffold and at least one main carrier. The at least one osteoconductive scaffold is made of a metal material, is manufactured by 3D printing, and has at least one connecting portion, at least one separation element, and a proliferation portion. The at least one connecting portion is porous. The at least one separation element is disposed on one of two sides of the at least one connecting portion. The proliferation portion is disposed on one of two sides of the at least one separation element away from the at least one connecting portion, wherein osteoblasts proliferate in the proliferation portion. The at least one main carrier is made of a medical macromolecular material and is mounted to the at least one connecting portion being porous, such that the at least one main carrier is mounted to the at least one osteoconductive scaffold.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view in partial section of a first embodiment of a human implant in accordance with the present invention;

FIG. 2 is a perspective view of an osteoconductive scaffold of the human implant in FIG. 1;

FIG. 3 is a bottom view of the osteoconductive scaffold of the human implant in FIG. 1;

FIG. 4 is a side view in partial section of the human implant along line 4-4 in FIG. 1;

FIG. 5 is a side view in partial section of the human implant along line 5-5 in FIG. 1;

FIG. 6 is a perspective view of a connecting element of a connecting portion of the osteoconductive scaffold of the human implant in FIG. 1;

FIG. 7 is a perspective view in partial section of a second embodiment of a human implant in accordance with the present invention;

FIG. 8 is a side view of a third embodiment of a human implant in accordance with the present invention;

FIG. 9 is a side view of a fourth embodiment of a human implant in accordance with the present invention;

FIG. 10 is a side view of a fifth embodiment of a human implant in accordance with the present invention;

FIG. 11 is an operational side view of a sixth embodiment of a human implant in accordance with the present invention;

FIG. 11A is an enlarged side view of FIG. 11;

FIG. 12 is a perspective view of a seventh embodiment of a human implant in accordance with the present invention;

FIG. 13 is a cross-sectional side view of an eighth embodiment of a human implant in accordance with the present invention; and

FIG. 13A is an enlarged cross-sectional side view of a proliferation portion of the osteoconductive scaffold of the human implant in FIG. 13.

### DETAILED DESCRIPTION OF THE PREFERRED EMBODIMENT

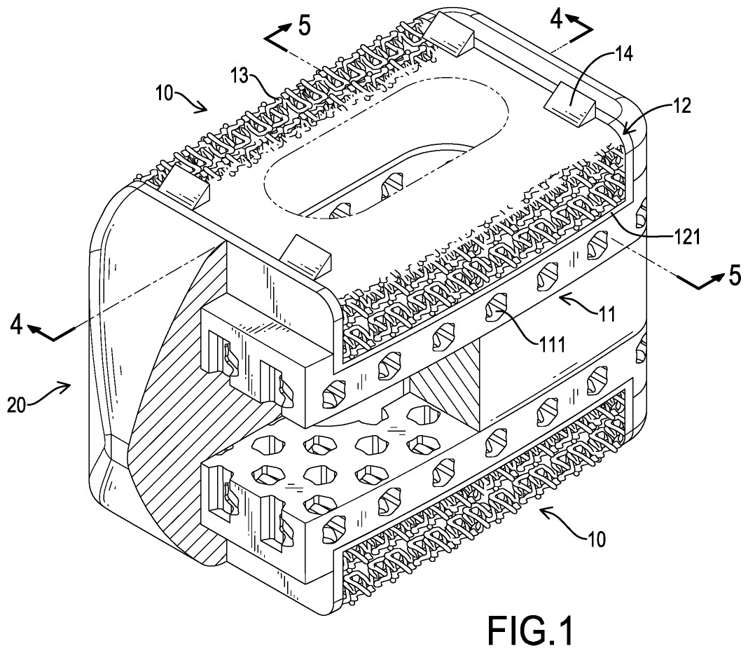

With reference to FIGS. 1, 4, and 5, a first embodiment of a human implant in accordance with the present invention is served as an interbody fusion cage and includes two osteoconductive scaffolds 10 and a main carrier 20.

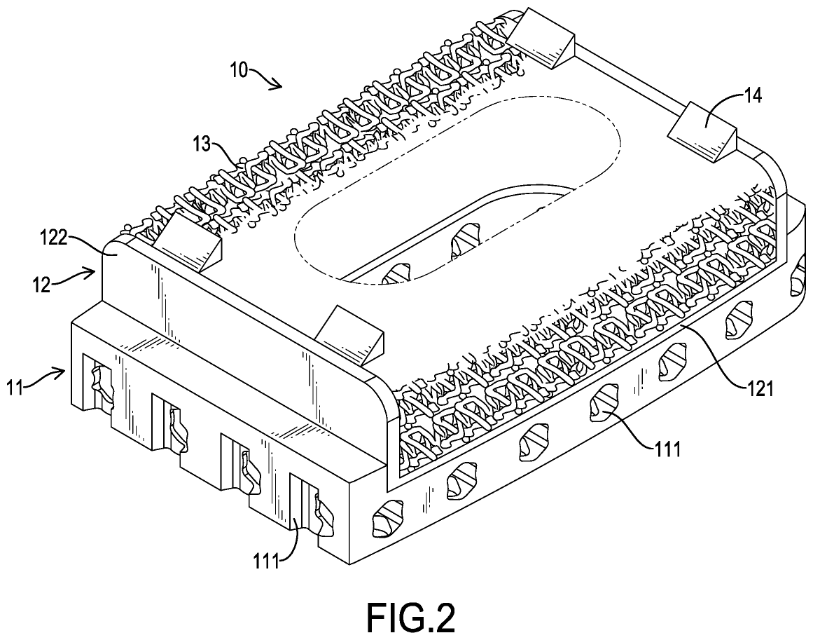

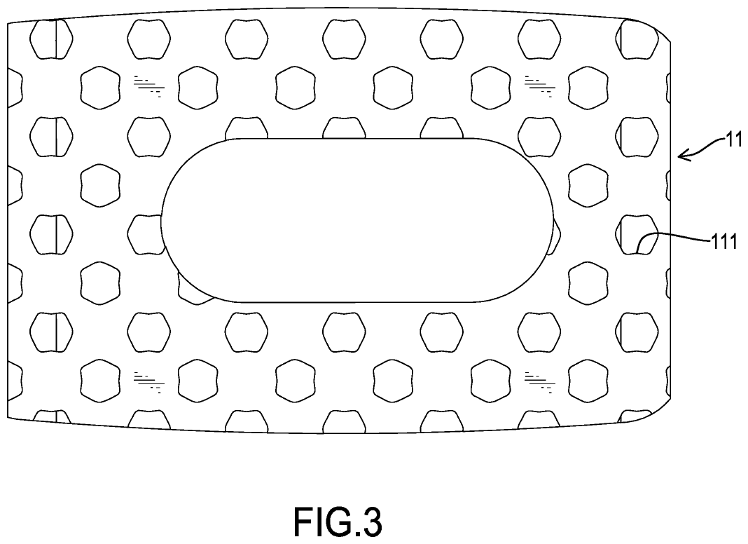

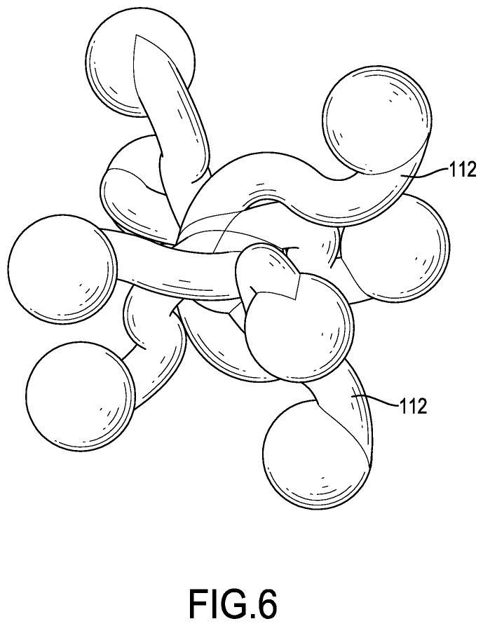

With reference to FIGS. 1, 4, and 5, the two osteoconductive scaffolds 10 are spaced apart and face opposite directions respectively. With reference to FIGS. 1 and 2, each one of the two osteoconductive scaffolds 10 is made of a metal material, is manufactured by 3D printing, which is also known as additive manufacturing, and has a connecting portion 11, a separation element 12, and a proliferation portion 13. With reference to FIGS. 1 to 3, the connecting portion 11 is porous, has a roughly rectangular section, and has multiple through holes 111 formed on an outer surface of the connecting portion 11 and an interior of the connecting portion 11. Furthermore, the connecting portion 11 may be a laminated structure with adjustable elastic modulus disclosed in TW Patent Pub No. 201946769. The laminated structure with adjustable elastic modulus has multiple connecting units. With reference to FIG. 6, each one of the multiple connecting units has multiple connecting elements 112, and each one of the multiple connecting elements 112 is an S-shaped curved component. The curvature of each one of the multiple connecting elements 112 can be individually adjusted to form different elastic moduli, so the connecting elements 112 at different positions of the connecting unit may have different curvatures. Moreover, the multiple connecting elements 112 can be combined with one another at different angles to meet the different requirements of tensile strength and compressive strength in different directions. Thus, the elastic modulus of the connecting portion 11 is adjustable.

With reference to FIGS. 1 and 2, the separation element 12 is disposed on one of two sides of the connecting portion 11 away from the other one of the two osteoconductive scaffolds 10 and has a base 121 and a lateral covering portion 122. The base 121 is disposed on one of two outer surfaces of the connecting portion 11 away from the other one of the two osteoconductive scaffolds 10. The lateral covering portion 122 extends from the base 121 and extends toward a direction away from the connecting portion 11. In the first embodiment of the human implant of the present invention, the lateral covering portion 122 has two side plates respectively disposed on two opposite edges of the base 121. The proliferation portion 13 is disposed on one of two sides of the base 121 of the separation element 12 away from the connecting portion 11, such that the base 121 is disposed between the connecting portion 11 and the proliferation portion 13. The lateral covering portion 122 partially covers a side periphery of the proliferation portion 13 via the two side plates thereof. The separation element 12 separates the connecting portion 11 and the proliferation portion 13.

In addition, the base 121 of the separation element 12 may be a solid component or a porous component with small-sized and densely populated pores. The proliferation portion 13 is porous to allow osteoblasts to proliferate therein, which is beneficial to the osseointegration of the human implant with two adjacent vertebrae after the human implant is implanted between the two adjacent vertebrae of the vertebral column. In such a way, the stability of the human implant of the present invention implanted into the vertebral column is enhanced, so the risk of subsidence of the human implant of the present invention implanted into the vertebral column is decreased. Moreover, the proliferation portion 13 may have a rough surface allowing the osteoblasts to proliferate thereon.

Furthermore, each one of the two osteoconductive scaffolds 10 is made of titanium with high tensile strength, remarkable corrosion resistance, and excellent biocompatibility, such that the proliferation of the osteoblasts in the proliferation portion 13 is promoted, and the blood vessels are also formed in the proliferation portion 13. In addition, with reference to FIGS. 1 and 2, each one of the two osteoconductive scaffolds 10 has at least one anti-migration element 14. The at least one anti-migration element 14 is disposed on one of two sides of the proliferation portion 13 away from the other one of the two osteoconductive scaffolds 10 and has an oblique surface. After the human implant of the present invention is implanted into the vertebral column, the friction forces between the human implant of the present invention and each one of the two adjacent vertebrae are increased, such that the stability of the human implant of the present invention implanted into the vertebral column is enhanced. The at least one anti-migration element 14 may be a solid structure or a porous structure. Under the circumstance that the at least one anti-migration element 14 is a porous structure, the osteoblasts can grow in the at least one anti-migration element 14. In the present invention, each one of the two osteoconductive scaffolds 10 has four said anti-migration elements 14 disposed on the proliferation portion 13 at spaced intervals. Two of the four said anti-migration elements 14 are adjacent to one of the two side plates of the lateral covering portion 122 of the separation element 12, and the other two of the four said anti-migration elements 14 are adjacent to the other one of the two side plates of the lateral covering portion 122 of the separation element 12.

With reference to FIGS. 1, 4, and 5, the main carrier 20 is made of a medical macromolecular material. The steps of the manufacture method of the first embodiment of the human implant in accordance with the present invention are as follows. First, place the two osteoconductive scaffolds 10 into a mold and make the proliferation portion 13 of each one of the two osteoconductive scaffolds 10 face opposite to where the other one of the two osteoconductive scaffolds 10 is located. Next, inject the medical macromolecular material forming the main carrier 20 into the mold by injection molding, such that the medical macromolecular material permeates into the multiple through holes 111 of the connecting portion 11 of each one of the two osteoconductive scaffolds 10. After the medical macromolecular material coagulates, the main carrier 20 is mounted to the two connecting portions 11 of the two osteoconductive scaffolds 10, making the two osteoconductive scaffolds 10 mounted to the main carrier 20 and disposed on two sides of the main carrier 20 respectively. Under the circumstance that the base 121 of the separation element 12 is a solid component, the medical macromolecular material is unable to permeate into the base 121 in the manufacturing process. Under the circumstance that the base 121 of the separation element 12 is a porous component with small-sized pores, the size of each one of the pores is too small for the medical macromolecular material to pass through.

With reference to FIGS. 1, 4, and 5, the main carrier 20 has an anterior portion 21 and a posterior portion 22. The anterior portion 21 is formed on one of two ends of the main carrier 20 and is bullet-shaped. The posterior portion 22 is formed on the other one of the two ends of the main carrier 20 and has a threaded hole 221 formed therein. In use, the threaded hole 221 of the main carrier 20 is threaded to an implantation tool to hold the human implant of the present invention in place. Next, align the anterior portion 21 of the main carrier 20 with an intervertebral space in need of implantation of an interbody fusion cage, and then the present invention can be directly implanted into the intervertebral space. The at least one anti-migration element 14 of each one of the two osteoconductive scaffolds 10 abuts against adjacent vertebra so as to increase the friction force between the human implant of the present invention and said adjacent vertebra, which lowers the risk of migration of the human implant of the present invention implanted into the intervertebral space. Moreover, the main carrier 20 may be made of a medical macromolecular material such as polyetheretherketone (PEEK), polyaryletheketone (PAEK), or ultra-high molecular weight polyethylene (UHMWPE). The elastic modulus of the medical macromolecular material is similar to that of the vertebral bone, lowering the risk of damage to said adjacent vertebra after the human implant of the present invention is implanted into the intervertebral space.

In addition, the main carrier 20 may be mounted to the two connecting portions 11 of the two osteoconductive scaffolds 10 by injection forming.

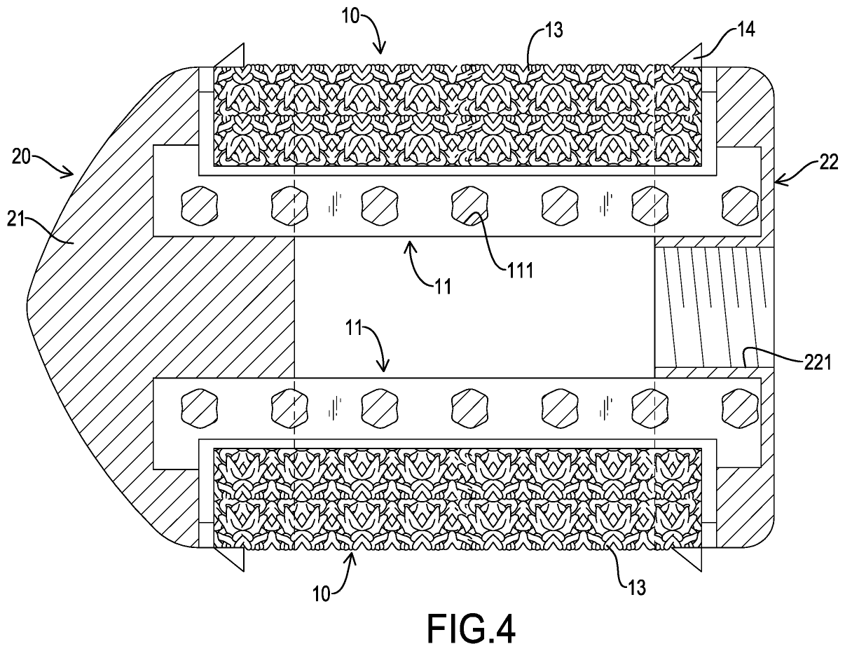

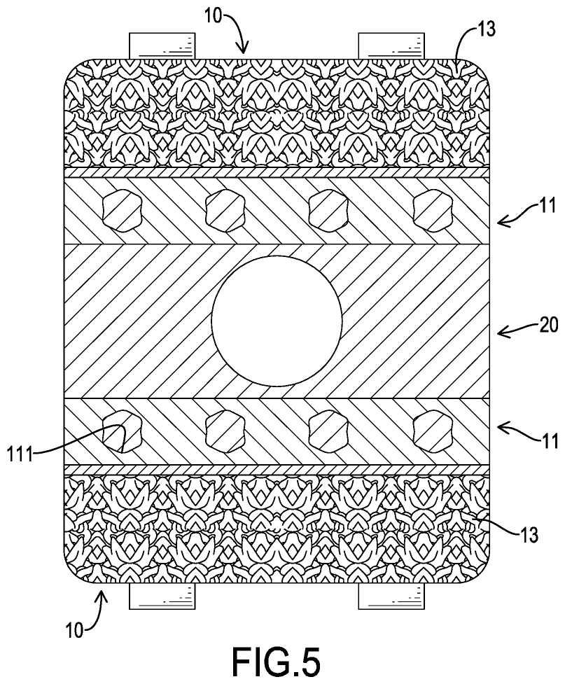

The connecting portion 11 of each one of the two osteoconductive scaffolds 10 is porous, so the medical macromolecular material forming the main carrier 20 can permeate into the connecting portion 11 before coagulating. After the medical macromolecular material coagulates and forms the main carrier 20, the main carrier 20 is firmly mounted to the two osteoconductive scaffolds 10. With reference to FIGS. 4 and 5, the section of the connecting portion 11 of each one of the two osteoconductive scaffolds 10 is roughly rectangular, so the positions in which the connecting portion 11 are in contact with the main carrier 20 are plane. In such a configuration, stress concentration is avoided, thereby preventing the separation of the main carrier 20 and each one of the two osteoconductive scaffolds 10 after a long-term use. With the structure that the proliferation portion 13 is porous or has a rough surface, the osteoblasts can proliferate in the proliferation portion 13 and the stress that the corresponding osteoconductive scaffold 10 can bear is decreased. Therefore, damage caused by the shielding effect to the two adjacent vertebrae can be avoided.

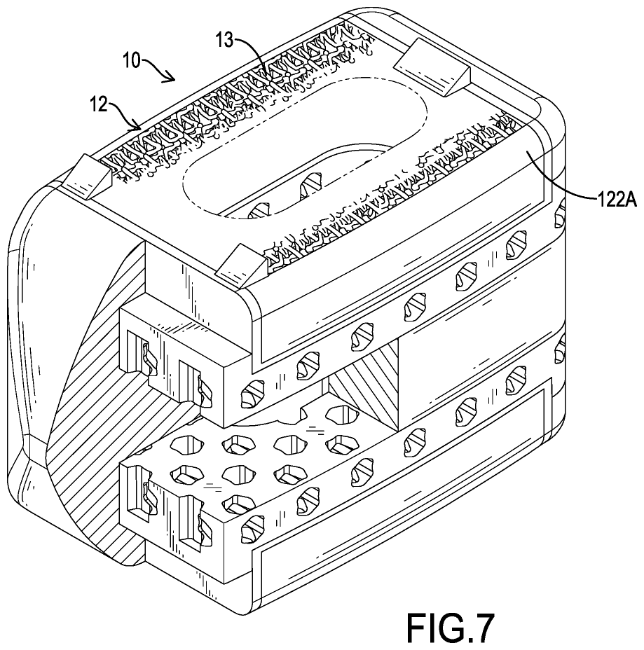

With reference to FIG. 7, a second embodiment of a human implant in accordance with the present invention is substantially the same as the first embodiment, and the difference between the second embodiment and the first embodiment is that: the lateral covering portion 122A of the separation element 12 of each one of the two osteoconductive scaffolds 10 is an annular closed loop, such that the lateral covering portion 122A completely covers the side periphery of the proliferation portion 13.

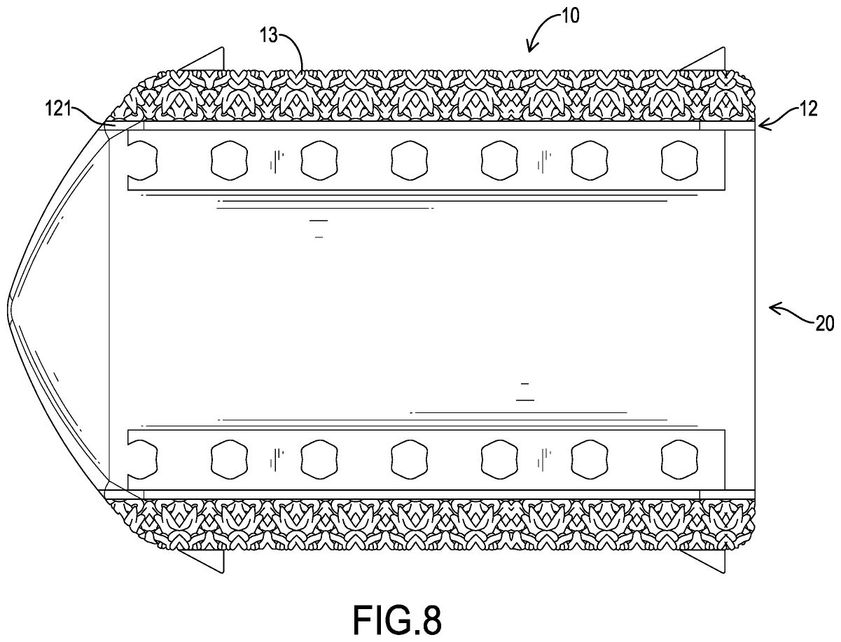

With reference to FIG. 8, a third embodiment of a human implant in accordance with the present invention is substantially the same as the first embodiment, and the difference between the third embodiment and the first embodiment is that: the separation element 12 of each one of the two osteoconductive scaffolds 10 does not have the lateral covering portion 122, and the base 121 of the separation element 12 separates the proliferation portion 13 and the main carrier 20.

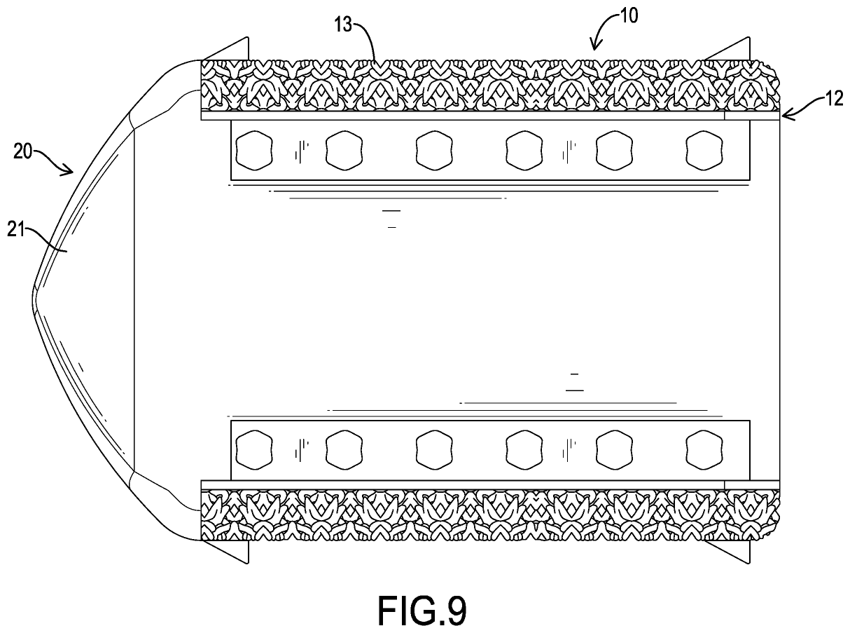

With reference to FIG. 9, a fourth embodiment of a human implant in accordance with the present invention is substantially the same as the first embodiment, and the difference between the fourth embodiment and the first embodiment is that: the separation element 12 of each one of the two osteoconductive scaffolds 10 does not have the lateral covering portion 122, and a portion of the main carrier 20 adjacent to the anterior portion 21 extends to positions that are flush with the rough surface of the proliferation portion 13 of each one of the two osteoconductive scaffolds 10.

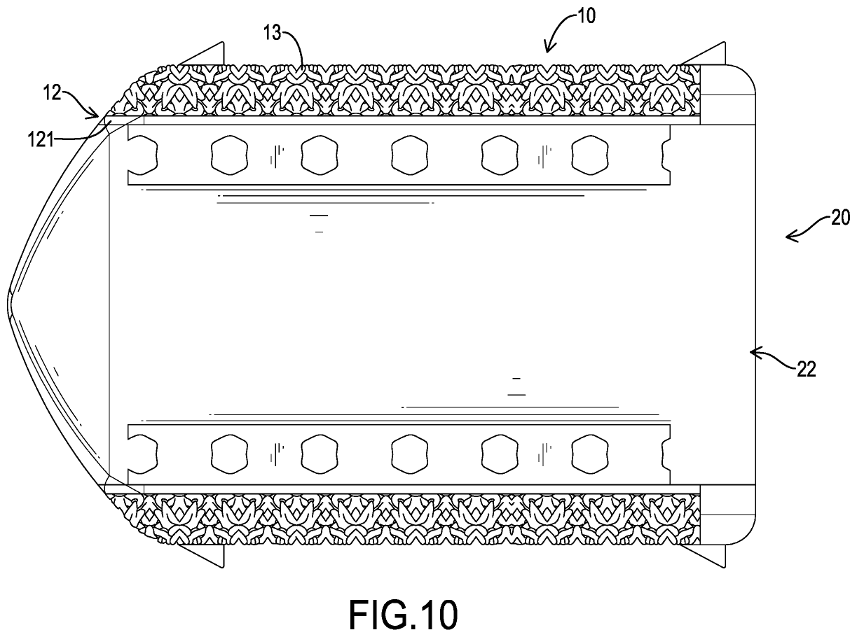

With reference to FIG. 10, a fifth embodiment of a human implant in accordance with the present invention is substantially the same as the first embodiment, and the difference between the fifth embodiment and the first embodiment is that: the separation element 12 of each one of the two osteoconductive scaffolds 10 does not have the lateral covering portion 122, and a portion of the main carrier 20 adjacent to the posterior portion 22 extends to positions that are flush with the surface of the proliferation portion 13 of each one of the two osteoconductive scaffolds 10.

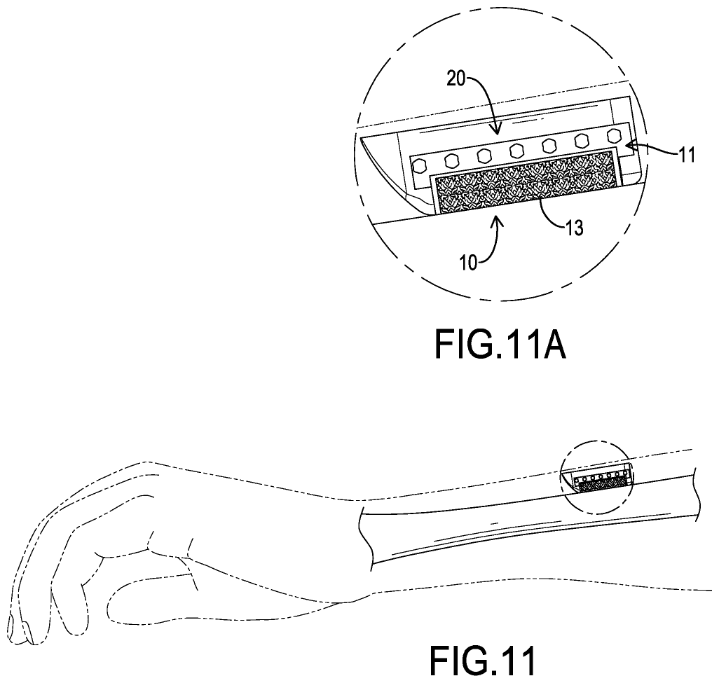

With reference to FIGS. 11 and 11A, a sixth embodiment of a human implant in accordance with the present invention is different from the first embodiment in that: the human implant of the sixth embodiment is served as a bone plate and includes only a single osteoconductive scaffold 10. The main carrier 20 is mounted to the connecting portion 11 of the osteoconductive scaffold 10. In use, the proliferation portion 13 of the osteoconductive scaffold 10 is attached to a fractured bone. After the osteoblasts proliferate in the proliferation portion 13, the human implant of the present invention served as a bone plate can gradually fuse with the bone at where the proliferation portion 13 of the osteoconductive scaffold 10 is attached to.

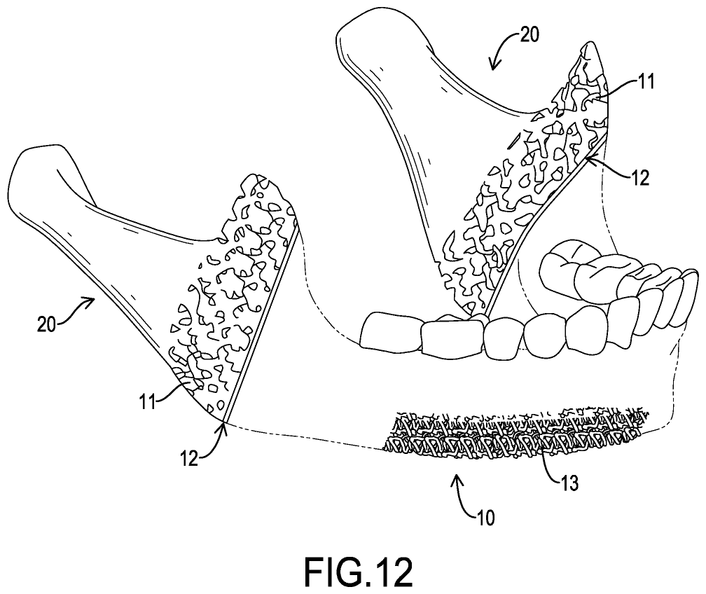

With reference to FIG. 12, a seventh embodiment of a human implant in accordance with the present invention is different from the first embodiment in that: the human implant of the seventh embodiment is served as an artificial mandible and includes two said main carriers 20 and only a single osteoconductive scaffold 10. The osteoconductive scaffold 10 is curved and has a proliferation portion 13, two said separation elements 12 and two said connecting portions 11. The proliferation portion 13 is curved. The two said separation elements 12 are respectively disposed on the two sides of the proliferation portion 13. The two said connecting portions 11 are respectively mounted to the two said separation elements 12, and each one of the two said connecting portions 11 is disposed on one of two sides of a respective one of the two said separation elements 12 away from the proliferation portion 13. The two said main carriers 20 are respectively mounted to the two said connecting portions 11 of the osteoconductive scaffold 10, and each one of the two said main carriers 20 is mounted to one of the two sides of the corresponding connecting portion 11 away from the corresponding separation element 12. Each one of the two said main carriers 20 is made of a medical macromolecular material, thereby preventing the direct contact and abrasion of the metal osteoconductive scaffold 10 with neighboring bones and muscles. Moreover, because the two said separation elements 12 and the proliferation portion 13 are all porous, the weight of the human implant of the seventh embodiment served as an artificial mandible is decreased.

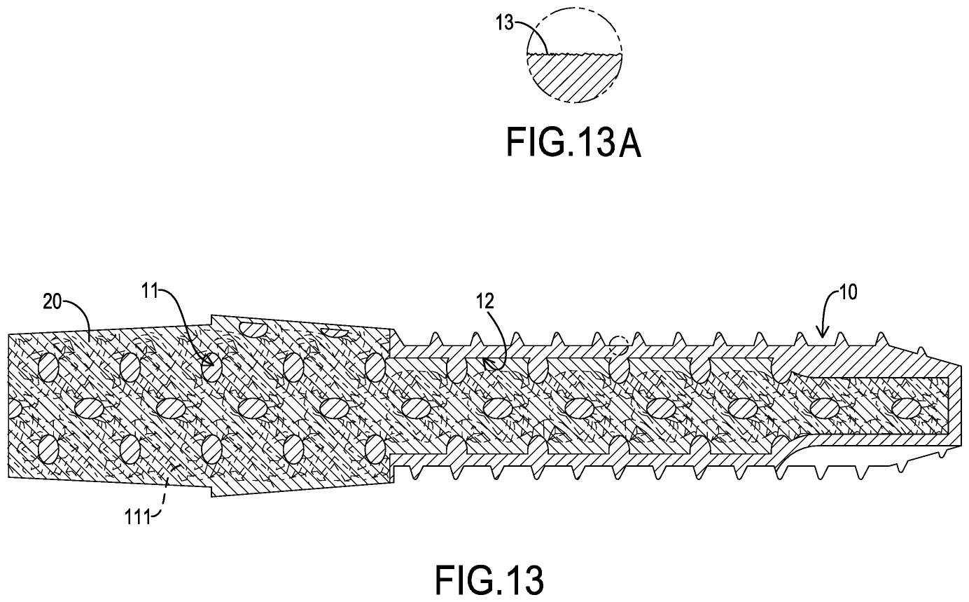

With reference to FIGS. 13 and 13A, an eighth embodiment of a human implant in accordance with the present invention is different from the first embodiment in that: the human implant of the eighth embodiment is served as an artificial tooth root and includes only a single osteoconductive scaffold 10. The osteoconductive scaffold 10 is a post and has an annular surface. The connecting portion 11 is disposed in a center of the osteoconductive scaffold 10 and has two sections and the multiple through holes 111 formed on the outer surface of the connecting portion 11 and the interior of the connecting portion 11. The separation element 12 is sleeved on one of the two sections of the connecting portion 11, such that the separation element 12 is disposed on one of the two sides of the connecting portion 11. A space is formed between the connecting portion 11 and the separation element 12. The proliferation portion 13 is sleeved on the separation element 12, such that the proliferation portion 13 is disposed on one of the two sides of the separation element 12 away from the connecting portion 11. The proliferation portion 13 has a thread formed on its surface and allows the osteoblasts to proliferate on the rough surface of the proliferation portion 13. The main carrier 20 is mounted to the connecting portion 11 of the osteoconductive scaffold 10, fills the space formed between the connecting portion 11 and the separation element 12 and the multiple through holes 111 of the connecting portion 11, and the main carrier 20 is sleeved on the other one of the two sections of the connecting portion 11 that is not sleeved by the separation element 12. The human implant of the eighth embodiment served as an artificial tooth root is implanted into the jawbone with the portion sleeved by the proliferation portion 13, such that the osteoblasts proliferate on the proliferation portion 13. The portion of the human implant of the eighth embodiment sleeved by the main carrier 20 partially extends out of the gum, and a dental crown is attached to the main carrier 20.

The connecting portion 11 and the proliferation portion 13 of the osteoconductive scaffold 10 in the first embodiment to the seventh embodiment are all porous, and such porous structure is hard to be made by traditional manufacturing methods. The technical feature that the main carrier 20 being tightly mounted to the connecting portion 11 of the osteoconductive scaffold 10 manufactured by the widely used 3D printing in recent years via injection molding or injection forming is critical to the present invention. With such a technical feature, a close combination of different materials can be achieved. In addition, porosity and sizes of pores in the proliferation portion 13 can be adjusted according to the health condition, the age, the habits, and the part of the body to be applied of a patient. In the eighth embodiment of the present invention, the proliferation portion 13 is not a porous structure, but the proliferation portion 13 has the rough surface allowing the osteoblasts to proliferate thereon.

With the aforementioned technical characteristics, the human implant in accordance with the present invention has the following advantages.

1. The proliferation portion 13 of the osteoconductive scaffold 10 in the present invention is either the porous structure manufactured by the gradually matured 3D printing technique in recent years or a component with the rough surface. In either case, the proliferation portion 13 facilitates the proliferation of the osteoblasts in the proliferation portion 13, and the extent of bone fusion of the present invention with adjacent bones is raised. Therefore, the risk of subsidence of the present invention served as the interbody fusion cage is decreased.

2. With the structure that the connecting portion 11 of the osteoconductive scaffold 10 is porous, the present invention prevents the circumstance that the osteoconductive scaffold 10 would separate from the main carrier 20 and the problem that the fatigue stress of the conventional human implant may exceed the endurance limit caused by the difference in elastic moduli between the metal connecting portion 11 and the medical macromolecular material.

3. Compared with the conventional human implant that needs the at least one fixation pin to be inserted through the at least one main carrier and the at least one osteoconductive scaffold, the medical macromolecular material forming the main carrier 20 permeates into the multiple through holes 111 of the connecting portion 11 manufactured by 3D printing technique via the injection molding technique or the injection forming technique. After the medical macromolecular material forming the main carrier 20 coagulates, the main carrier 20 is tightly mounted to the connecting portion 11 of the osteoconductive scaffold 10 without any fixation elements.

4. As mentioned in point 3, the tightness between the main carrier 20 and the osteoconductive scaffold 10 is higher than that of the components of the conventional human implant since the main carrier 20 is mounted to the osteoconductive scaffold 10 manufactured by 3D printing via the injection molding or injection forming. The main carrier 20 is hard to separate from the osteoconductive scaffold 10, and the life expectancy of the present invention is therefore extended. Moreover, the present invention solves the problems of the conventional human implant that the at least one fixation pin tends to detach and remains in the human body.

5. Since the connecting portion 11 of the osteoconductive scaffold 10 is either the porous structure porous or a component with the rough surface, the stress that the osteoconductive scaffold 10 can bear is decreased. Thereby, damage caused by the shielding effect to the two adjacent vertebrae when the present invention is served as an interbody fusion cage can be avoided.

6. The section of the connecting portion 11 of the osteoconductive scaffold 10 is roughly rectangular, so the positions in which the connecting portion 11 are in contact with the main carrier 20 are plane. In such a configuration, stress concentration is avoided, thereby preventing the separation of the main carrier 20 and the osteoconductive scaffold 10 after a long-term use.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A human implant comprising:
at least one osteoconductive scaffold being made of a metal material, manufactured by 3D printing, and having
at least one connecting portion being porous;
at least one separation element disposed on one of two sides of the at least one connecting portion; and
a proliferation portion disposed on one of two sides of the at least one separation element away from the at least one connecting portion, wherein osteoblasts proliferate in the proliferation portion; and
at least one main carrier being made of a medical macromolecular material and mounted to the at least one connecting portion being porous, such that the at least one main carrier is mounted to the at least one osteoconductive scaffold.

2. The human implant as claimed in claim 1, wherein the proliferation portion of the at least one osteoconductive scaffold is porous.

3. The human implant as claimed in claim 1, wherein the proliferation portion of the at least one osteoconductive scaffold has a rough surface.

4. The human implant as claimed in claim 1, wherein the at least one connecting portion of the at least one osteoconductive scaffold has multiple connecting units, and each one of the multiple connecting units has
multiple connecting elements, and each one of the multiple connecting elements being a curved component, such that the elastic modulus of the at least one connecting portion of the at least one osteoconductive is adjustable.

5. The human implant as claimed in claim 2, wherein the human implant has two said osteoconductive scaffolds mounted to two sides of the at least one main carrier respectively, and the at least one main carrier is mounted to the at least one connecting portion of each one of the two said osteoconductive scaffolds.

6. The human implant as claimed in claim 3, wherein the human implant has two said osteoconductive scaffolds mounted to two sides of the at least one main carrier respectively, and the at least one main carrier is mounted to the at least one connecting portion of each one of the two said osteoconductive scaffolds.

7. The human implant as claimed in claim 4, wherein the human implant has two said osteoconductive scaffolds mounted to two sides of the at least one main carrier respectively, and the at least one main carrier is mounted to the at least one connecting portion of each one of the two said osteoconductive scaffolds.

8. The human implant as claimed in claim 1, wherein the at least one separation element of the at least one osteoconductive scaffold has a base disposed between the at least one connecting portion and the proliferation portion.

9. The human implant as claimed in claim 2, wherein the at least one separation element of the at least one osteoconductive scaffold has a base disposed between the at least one connecting portion and the proliferation portion.

10. The human implant as claimed in claim 3, wherein the at least one separation element of the at least one osteoconductive scaffold has a base disposed between the at least one connecting portion and the proliferation portion.

11. The human implant as claimed in claim 4, wherein the at least one separation element of the at least one osteoconductive scaffold has a base disposed between the at least one connecting portion and the proliferation portion.

12. The human implant as claimed in claim 5, wherein the at least one separation element of each one of the two said osteoconductive scaffolds has a base disposed between the at least one connecting portion and the proliferation portion.

13. The human implant as claimed in claim 6, wherein the at least one separation element of each one of the two said osteoconductive scaffolds has a base disposed between the at least one connecting portion and the proliferation portion.

14. The human implant as claimed in claim 7, wherein the at least one separation element of each one of the two said osteoconductive scaffolds has a base disposed between the at least one connecting portion and the proliferation portion.

15. The human implant as claimed in claim 8, wherein the base of the at least one separation element of the at least one osteoconductive scaffold is a solid component.

16. The human implant as claimed in claim 9, wherein the base of the at least one separation element of the at least one osteoconductive scaffold is a solid component.

17. The human implant as claimed in claim 10, wherein the base of the at least one separation element of the at least one osteoconductive scaffold is a solid component.

18. The human implant as claimed in claim 11, wherein the base of the at least one separation element of the at least one osteoconductive scaffold is a solid component.

19. The human implant as claimed in claim 8, wherein the base of the at least one separation element of the at least one osteoconductive scaffold is a porous component that prevents permeation of the medical macromolecular material.

20. The human implant as claimed in claim 9, wherein the base of the at least one separation element of the at least one osteoconductive scaffold is a porous component that prevents permeation of the medical macromolecular material.

21. The human implant as claimed in claim 10, wherein the base of the at least one separation element of the at least one osteoconductive scaffold is a porous component that prevents permeation of the medical macromolecular material.

22. The human implant as claimed in claim 11, wherein the base of the at least one separation element of the at least one osteoconductive scaffold is a porous component that prevents permeation of the medical macromolecular material.

23. The human implant as claimed in claim 12, wherein the base of the at least one separation element of each one of the two said osteoconductive scaffolds is a solid component.

24. The human implant as claimed in claim 13, wherein the base of the at least one separation element of each one of the two said osteoconductive scaffolds is a solid component.

25. The human implant as claimed in claim 14, wherein the base of the at least one separation element of each one of the two said osteoconductive scaffolds is a solid component.

26. The human implant as claimed in claim 12, wherein the base of the at least one separation element of each one of the two said osteoconductive scaffolds is a porous component that prevents permeation of the medical macromolecular material.

27. The human implant as claimed in claim 13, wherein the base of the at least one separation element of each one of the two said osteoconductive scaffolds is a porous component that prevents permeation of the medical macromolecular material.

28. The human implant as claimed in claim 14, wherein the base of the at least one separation element of each one of the two said osteoconductive scaffolds is a porous component that prevents permeation of the medical macromolecular material.

29. The human implant as claimed in claim 2, wherein
the at least one osteoconductive scaffold has
two said separation elements respectively disposed on two sides of the proliferation portion; and
two said connecting portions respectively mounted to the two said separation elements, and each one of the two said connecting portions disposed on one of the two sides of a respective one of the two said separation elements away from the proliferation portion; and
the human implant comprises two said main carriers respectively mounted to the two said connecting portions of the at least one osteoconductive scaffold, and each one of the two said main carriers is mounted to one of the two sides of the corresponding connecting portion away from the corresponding separation element.

30. The human implant as claimed in claim 3, wherein
the at least one osteoconductive scaffold has
two said separation elements respectively disposed on two sides of the proliferation portion; and
two said connecting portions respectively mounted to the two said separation elements, and each one of the two said connecting portions disposed on one of the two sides of a respective one of the two said separation elements away from the proliferation portion; and
the human implant comprises two said main carriers respectively mounted to the two said connecting portions of the at least one osteoconductive scaffold, and each one of the two said main carriers is mounted to one of the two sides of the corresponding connecting portion away from the corresponding separation element.

31. The human implant as claimed in claim 4, wherein
the at least one osteoconductive scaffold has
two said separation elements respectively disposed on two sides of the proliferation portion; and
two said connecting portions respectively mounted to the two said separation elements, and each one of the two said connecting portions disposed on one of the two sides of a respective one of the two said separation elements away from the proliferation portion; and
the human implant comprises two said main carriers respectively mounted to the two said connecting portions of the at least one osteoconductive scaffold, and each one of the two said main carriers is mounted to one of the two sides of the corresponding connecting portion away from the corresponding separation element.

32. The human implant as claimed in claim 1, wherein
the at least one osteoconductive scaffold is a post;
the at least one connecting portion is disposed in a center of the at least one osteoconductive scaffold and has two sections;
the at least one separation element is sleeved on one of the two sections of the at least one connecting portion, such that the at least one separation element is disposed on one of the two sides of the at least one connecting portion;
the proliferation portion is sleeved on the at least one separation element and has a rough surface, such that the proliferation portion is disposed on one of the two sides of the at least one separation element away from the at least one connecting portion; and
the at least one main carrier is mounted to the at least one connecting portion of the at least one osteoconductive scaffold and is sleeved on the other one of the two sections of the at least one connecting portion.

33. The human implant as claimed in claim 4, wherein
the at least one osteoconductive scaffold is a post;
the at least one connecting portion is disposed in a center of the at least one osteoconductive scaffold and has two sections;
the at least one separation element is sleeved on one of the two sections of the at least one connecting portion, such that the at least one separation element is disposed on one of the two sides of the at least one connecting portion;
the proliferation portion is sleeved on the at least one separation element and has a rough surface, such that the proliferation portion is disposed on one of the two sides of the at least one separation element away from the at least one connecting portion; and
the at least one main carrier is mounted to the at least one connecting portion of the at least one osteoconductive scaffold and is sleeved on the other one of the two sections of the at least one connecting portion.
